(19) United States
(12) Patent Application Publication
(10) Pub. No.: US 2024/0157075 A1
(43) Pub. Date: May 16, 2024

(54) ENDOTRACHEAL TUBE DEVICE

(71) Applicants: Basharat BUCHH, Granger, IN (US); Sabia JAN, Kashmir (IN)

(72) Inventors: Basharat BUCHH, Granger, IN (US); Sabia JAN, Kashmir (IN)

(21) Appl. No.: 18/207,092

(22) Filed: Jun. 7, 2023

Related U.S. Application Data
(60) Provisional application No. 63/426,020, filed on Nov. 16, 2022.

(51) Int. Cl. A61M 16/04

(52) U.S. Cl. CPC A61M 16/0402; A61M 16/0445; A61M 2025/0008

(57) ABSTRACT

An endotracheal tube device is described herein. The device includes a tubular body having a proximal end and a distal end opposite the proximal end, the distal end having an opening for ventilation, and a distal tip. The device further includes a cuff disposed around the tubular body and configured to be inflated to seal the cuff against a wall of a trachea, the cuff being pear-shaped and having a tapered proximal end and a distal end opposite the proximal end. The device further includes a plurality of markings disposed on an outer surface of the tubular body. This configuration allow the device to be placed in the trachea such that the cuff is positioned below the cricoid of the trachea and prevents the chance of pressure injury if the cuff is positioned in the cricoid.

### CROSS REFERENCE TO RELATED APPLICATIONS

This application claims benefit under 35 U.S.C. § 119(e) of U.S. Provisional Application No. 63/426,020 filed on Nov. 16, 2022, the entire contents of all of which are incorporated herein by reference.

### TECHNICAL FIELD

The present disclosure relates to an endotracheal tube device.

### BACKGROUND

Endotracheal tubes are flexible plastic tubes used for mechanical ventilation in a patient's trachea during intubation. For pediatric intubation, current endotracheal tubes typically include a long hollow tube (usually made of PVC) with a balloon at the distal end (i.e. a cuff or distal cuff) and a tube tip. The practice of pediatric intubation has changed from non-cuffed to cuffed endotracheal tube use due to multiple advantages. The distal cuff prevents the tube from moving, seals the airway from oropharyngeal contents, and prevents air leak.

However, the cuff has been implicated in pressure-related injuries to the pediatric airway especially if it is placed in less-than-ideal location. The ideal position of a cuffed endotracheal tube in pediatric patients as known in the art is one which guarantees cuff-free subglottic airway, specifically the location below the cricoid has been advocated as the ideal position of the endotracheal tube cuff, in order to prevent cuff-related pressure effects. To ensure that the cuff is placed below the cricoid in real-time, ultrasound has been used in real-time and has been an effective tool to determine the cuff placement.

Current endotracheal tubes used for children come with varying cuff shapes and a variable cuff location on the shaft of the endotracheal tube. The distal cuff is ideally positioned below the cricoid ring within the trachea, while the endotracheal tube tip is ideally positioned proximal to the carina within the trachea. However, a common problem after intubation is suboptimal placement of the cuff within the trachea, typically at or above the cricoid ring, which causes airway injury. Because the cricoid ring is the only fixed dimension in the pediatric airway, presence of an inflated cuff in this location can result in pressure injury to the surrounding tissue. In addition, cuff location in current endotracheal tubes is too close to the vocal-cord marker, leading to a significant risk of an inflated cuff being positioned within the cricoid, even though the tip of the tube is ideally located within the trachea.

There has been focus across the industry on placing the markers on the tracheal tube shaft of endotracheal tubes. Current markers on endotracheal tubes guide the depth of intubation, ensuring that the tip of the endotracheal tube is placed above the carina without taking cuff placement into consideration. These markings have no role in proper positioning of the endotracheal tube cuff, however. In addition, there is variability with regards to the location and number of the markers on current endotracheal tubes with a lack of clarity or purpose.

### SUMMARY

There is therefore a need for an endotracheal tube device that easily positions the cuff below the cricoid area and prevents the chance of pressure injury if the cuff is positioned in the cricoid. The present disclosure is a device for the trachea. The device includes a tubular body having a proximal end and a distal end, with the distal end having A) an opening for ventilation, and B) a distal tip. The device further includes a cuff disposed around the tubular body and configured to be inflated to seal the cuff against a wall of a trachea. The cuff is frustrum-shaped with a tapered end directed proximally. The device further includes markings disposed on an outer surface of the tubular body. The markings are configured to allow the device to be placed in the trachea such that the cuff is positioned below the cricoid of the trachea when the marking is placed at the level of vocal cords at the time of endotracheal tube placement.

### BRIEF DESCRIPTION OF THE DRAWINGS

The foregoing summary, as well as the following detailed description of illustrative embodiments of the present application, will be better understood when read in conjunction with the appended drawings. For the purposes of illustrating the present application, there is shown in the drawings illustrative embodiments of the disclosure. It should be understood, however, that the application is not limited to the precise arrangements and instrumentalities shown. In the drawings:

FIG. 1 is a schematic view of an endotracheal tube device according to an embodiment of the present disclosure;

FIG. 2 is a perspective view of an endotracheal tube device according to an embodiment of the present disclosure;

FIG. 3 is a side view of a distal portion of the endotracheal tube device shown in FIG. 2;

FIG. 4 is a diagrammatic representation of the endotracheal tube device shown in FIGS. 2-3 inserted into a trachea; and

FIG. 5 is a chart depicting distribution of patients in a study based on size of an endotracheal tube device shown in FIGS. 1-4.

### DETAILED DESCRIPTION OF ILLUSTRATIVE EMBODIMENTS

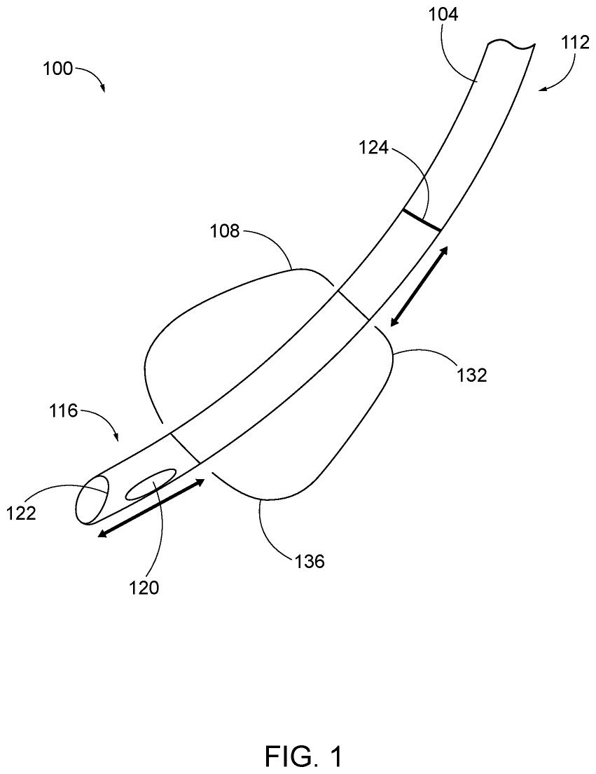

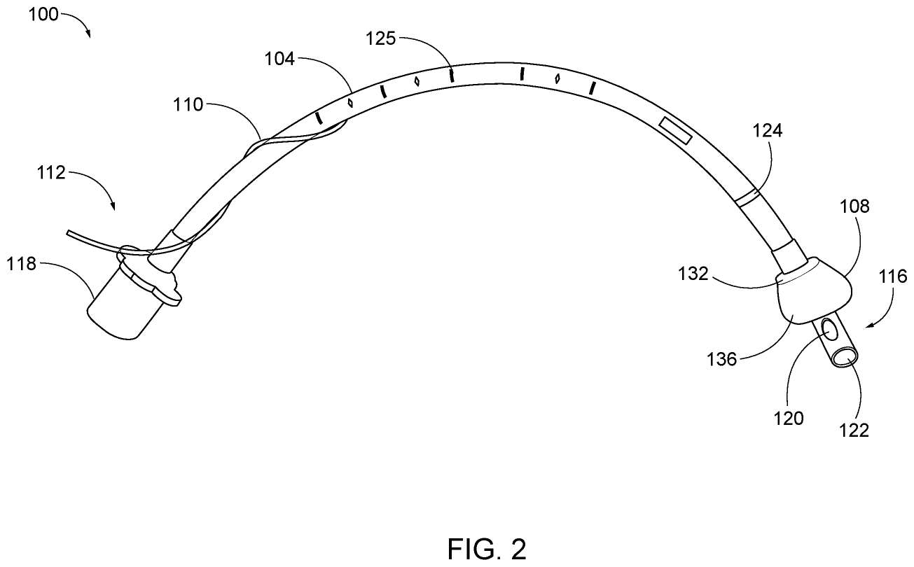

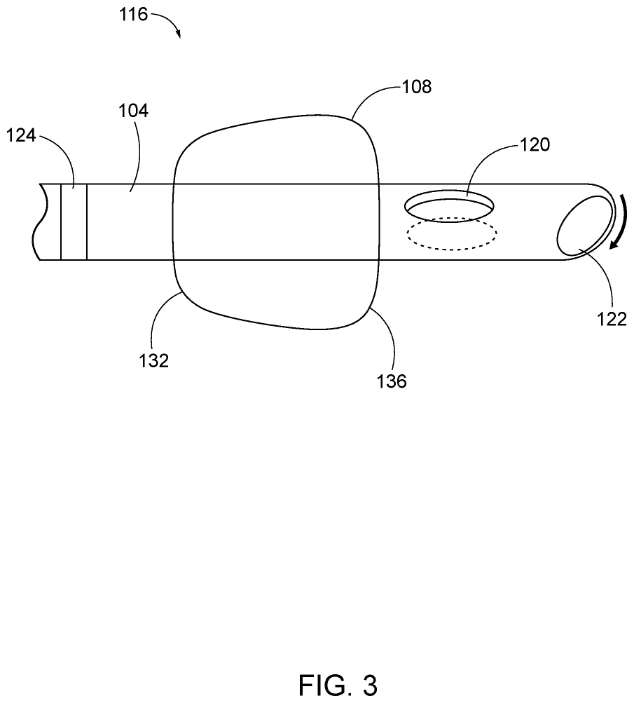

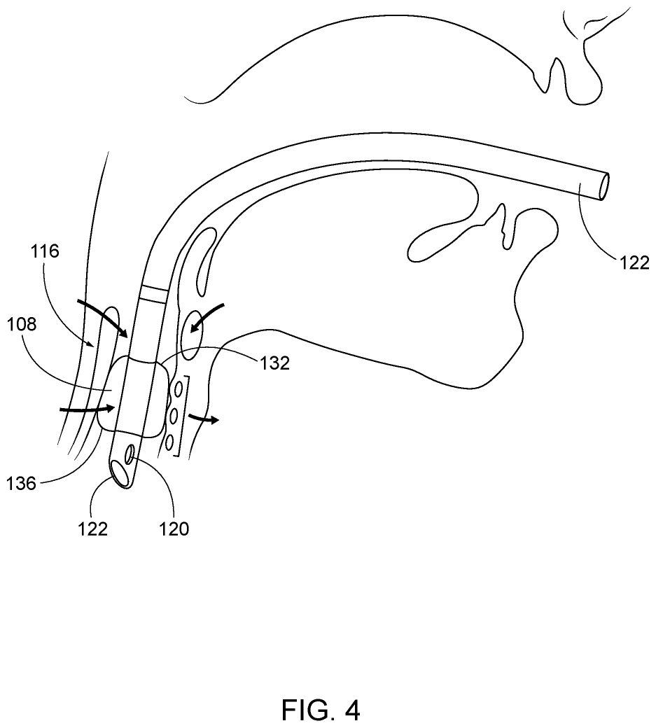

Referring to FIGS. 1-4, embodiments of the present disclosure include a device 100 for a patient's trachea having a tubular body 104 and a cuff 108 coupled to the tubular body 104. The device 100 is a sterile, single use device configured to be inserted in the trachea for ventilation. The device 100 easily positions the cuff 108 below the cricoid area and prevents the chance of pressure injury if the cuff 108 is positioned in the cricoid. In the illustrated embodiments of the present disclosure, the patient is a pediatric patient. In other embodiments, the patient may be an infant, adult, or geriatric patient. In one embodiment, the device 100 may further include an inflation line 110, a pilot balloon (not depicted) coupled to a proximal end of the inflation line, and a one-way valve coupled to a proximal end of the pilot balloon.

The tubular body 104 includes a proximal end 112 and a distal end 116 opposite the proximal end 112. The tubular body 104 is hollow to allow for air to be ventilated through the tubular body. The tubular body 104 includes a length L. The length L varies between 18.0 cm to 26.0 cm. The tubular body 104 further includes an inner diameter ID and an outer diameter OD. The inner diameter ID is configured to vary in size based on the size of the trachea. The size of the inner diameter ranges from about 3.0 mm to about 6.0 mm. The size of the outer diameter ranges from about 4.2 mm to about 8.2 mm.

The tubular body 104 is flexible and is shaped to have a standard Magill curve of 140 degrees, as is known in the art. In the illustrated embodiment, the tubular body 104 is made from Polyvinyl chloride (PVC) resin. In alternative embodiments, the tubular body 104 is made of various materials including rubber and polyurethane.

The proximal end 112 is configured to be coupled to a connector 118. The connector 118 is configured to be connected to a Y-piece of a breathing circuit, a ventilation bag, for example an Ambu bag, or to a ventilator tubing. The connector 118 is sized and shaped to provide various connections between breathing circuits, ventilation bags, or ventilation tubing. In the illustrated embodiment, the connector 118 has a standard diameter of 15.0 mm. In other embodiments, the dimensions of the connector diameter may vary.

The distal end 116 is configured to be inserted into a patient's trachea. The distal end 116 includes an opening 120 for ventilation and a blunt distal tip 122 spaced from the opening 120. The opening 120 is configured to provide an alternate ventilator port in case the distal tip 122 becomes blocked when inserted in the trachea. In the illustrated embodiment, the distal tip 122 is tapered. In alternative embodiments, the distal tip 122 has a straight edge or is enlarged.

In the illustrated embodiment, the tubular body 104 includes a vocal cord marker 124 disposed on the outer surface of the tubular body 104 to guide the depth of insertion of the tubular body 104 into a patient. Currently available endotracheal tubes include a vocal cord marker, which rest at the vocal cord when placing endotracheal tube in a patient. Current markers are devised to ensure that the endotracheal tube stays above the carina, i.e. the branching point of the patient's main airway, to ensure that both lungs are ventilated. These current markers, however, are not devised to assure ideal positioning of a cuff in the trachea.

In the illustrated embodiment, the marker 124 is configured to ensure that the cuff 108 stays below the cricoid outlet of a patient while the distal tip 122 stays above the carina of the patient. The marker 124 is further configured to allow the cuff 108 to be positioned and sit below the cricoid ring and allow the distal tip 122 to be positioned above the patient's carina, thus preventing the cuff 108 from being positioned in the cricoid area. This configuration minimizes the chance of endobronchial intubation.

The marker 124 depicts a vocal cord marking based on anatomical measurements of pediatric patient airways. The marker 124 may be customized to fit a particular endotracheal tube size. The marker 124 may have a width of about 10.0-15.0 mm. The distance from a proximal end of marker to the to the distal tip 122 may range from about 28.0 mm to about 66.0 mm based on patient characteristics.

In addition, the tubular body 104 includes additional markings 125 positioned along the length of the tubular body 104. The additional markings 125 are further configured to identify the exact or precise position of the tubular body 104 at the mouth, lips, or teeth of the patient. In one embodiment, the tubular body 104 further includes a radiopaque line (not depicted) along the full length of the tubular body 104.

The cuff 108 is disposed around the tubular body 104. The cuff 108 is configured to be inflated to seal the cuff 108 against a wall of a patient's trachea. Current cuffs are either olive or cylindrical in shape; however, these current cuff shapes are disadvantageous. Olive cuffs taper on both upper and lower sides of the tube, preventing pressure from being evenly distributed on the airway mucosa. In addition, olive cuffs allow maximal pressure to occur at the point of maximal diameter, making the area of contact more prone to injury. Cylindrical cuffs allow the cuff to hang above and below the attachments on the tube, increasing the chances of the cuff extending into the cricoid upon inflation.

In the illustrated embodiment, the cuff 108 is frustrum-shaped. The cuff 108 includes a proximal tapered end 132 and a distal end 136 that is larger than the proximal end 132. This configuration prevents the chance of pressure injury in the rare occurrence of the cuff being positioned in the cricoid, as it allows low volume and pressure in the tapered portion of the cuff.

In the illustrated embodiment, the cuff 108 is made of micro-thin polyurethane. In this configuration, the cuff 108 is thin-walled. In one embodiment, the thickness of the cuff 108 is 10.0-12.0 microns. The thin wall configuration allows the thin polypropylene cuff membrane to seal the patient's airway at lower pressures (e.g., 12.0-15.0 cm H₂O). The thin material permits a high volume, low pressure (“HVLP”) cuff to reduce cuff pressure at an average cuff pressure of 15.0 cm H₂O. This cuff pressure is below the capillary perfusion pressure in the mucosal lining of the airway, minimizing the risk of perfusion injury to the tracheal mucosal wall. Furthermore, in the rare case of the cuff 108 being too high in the airway, the tapered part of the cuff 108 is configured to be positioned in the cricoid area, further minimizing the risk of pressure injury to the surrounding tissue as cuff expansion is away from the cricoid.

The length of the cuff 108 may range from about 8.0 mm to about 18.0 mm. The distance between the markings 124 and the cuff proximal end 132 may range from about 12.0 mm to about 28.0 mm. The distance between the markings 124 and the cuff distal end 136 may range from about 20.0 mm. to about 46.0 mm. The distance between the cuff distal end 136 and the distal tip 122 may range from about 8.0 mm to about 20.0 mm.

Proper vocal cord marking on endotracheal tubes with standardized age-specific measurements of the airway would assure placement of the endotracheal tube cuff below the cricoid ring (cricoid outlet), thus making intubation safer and better-tolerated. As a result, pediatric airway shape and dimensions were studied in order to develop measurements for the marker 124.

Data was obtained from descriptions of pediatric airway shape and dimensions as described by Eckenhoff J E, Some anatomic considerations of the infant larynx influencing endotracheal anesthesia. Anesthesiology 1951; 12:401-410; Bayeux R. Tubage de larynx dans le croup. Presse Medicale 1897; 6:29-33; Wani T M, Bissonnette B, Malik M R, Ramesh A, Hayes D, Al Sohaibani M, Tobias J D. Age-based analysis of pediatric upper airway dimensions using computed tomography imaging. Pediatric Pulmonology 2016; 51:267-271; Wani T M, John J, Rehman S, Bhaskar P, Sahabudheen A F, Mahfoud Z R, Tobias J D. Point of care ultrasound to confirm endotracheal tube cuff position in relationship to the cricoid in the pediatric population. Pediatric Anesthesia 2021; 31(12):1310-1315; Litman R S, Weissend E E, Shibata D, Westesson P L. Developmental changes of laryngeal dimensions in unparalyzed, sedated children. Anesthesiology 2003; 98:41-45; Holzki J F, Laschat M, Puder C. The Pediatric Larynx: A Complicated Organ. Anesthesia Analgesia 2010; 110:1509-10; G. J. Noback, The lineal growth of the respiratory system during fetal and neonatal life as expressed by graphic analysis and empirical formulae. American Journal of Anatomy 1925; 36:235-273; J. Holzki, K. A. Brown, R. G. Carroll, C. J. Cote, The anatomy of the pediatric airway: has our knowledge changed in 120 years? A review of historic and recent in-vestigations of the anatomy of the pediatric larynx. Paediatric Anaesthesia 2018; 28:13-22; Propst E J, Gorodensky J H, Wolter N E. Length of the Cricoid and Trachea in Children: Predicting Intubation Depth to Prevent Subglottic Stenosis. Laryngoscope 2021; 00:1-10; Dave M H, Kemper M, Schmidt A R, Both C P, Weiss M. Pediatric airway dimensions—a summary and presentation of existing data. Paediatric Anaesthesia 2019; 29:782-789. The measurements were based on cadaveric airways as well as in vivo, using calibrating rods (in cadavers), bronchoscopy, computed tomography (CT) images, MRI images and ultrasound modalities, and data from pediatric anesthesia, pediatric pulmonology, pediatric otorhinolaryngology based on 2-dimensional and 3-dimensional CT image-based data, and ultrasound based studies.

Using data from previous studies as guide, we calculated ideal positioning of vocal cord markings on our endotracheal tubes. We used various measurements including vocal cord to cricoid outlet dimensions, vocal cord to carina measurements, the cricoid and tracheal dimensions to derive ideal vocal cord to proximal cuff distances (lengths). We formulated and conducted the study, focusing on the cuff placement below the cricoid, and efficacy of the vocal cord markers. The pilot study checking the efficacy of the vocal cord markers (ensuring that the proximal end of the cuff stayed below the cricoid), we achieved excellent results with endotracheal tube-cuff below cricoid in 96% patients.

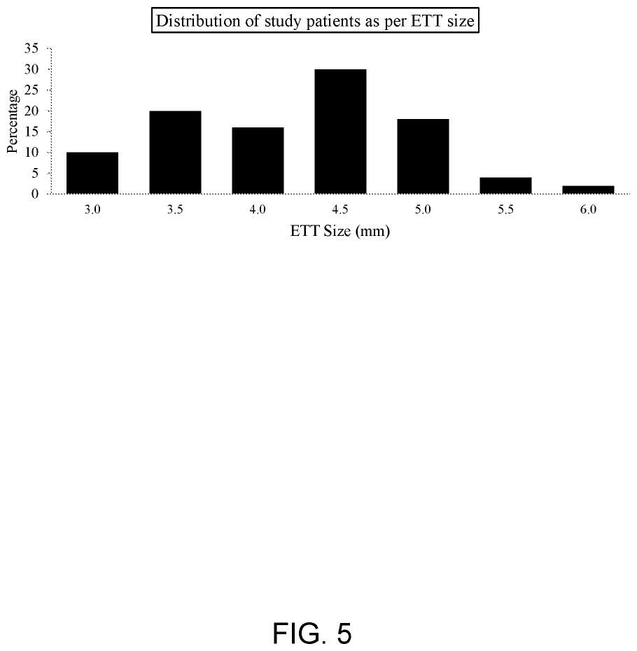

To confirm the accuracy of measurements of the marker 124 for various ages, a study was conducted that utilized the device 100 with marker 124 to check the cuff 108 position in relation to the cricoid ring of patients. The study included 50 pediatric patients between the ages of 1 day and 10 years old. Each patient was intubated with the device 100. The vocal cord marker 124 was placed at the level of each patient's vocal cords. Placement of the cuff 108 was then verified by real-time ultrasound. FIG. 5 depicts the distribution of the patients as per the different sizes of the device. The association of the cuff 108 position of the various sizes of the device in relation to the cricoid ring and tracheal rings are shown in Table 1, below.

TABLE 1
Association of ETT (device 100) size with USG Position in study patients
Association of ETT size with USG Position in study patients

| | Cuff position (Ultrasound based) | | | | | | |
|---|---|---|---|---|---|---|---|
| | | T1 (first | T2 (second | T3 (third | T4 (fourth | | |
| ETT | | tracheal | tracheal | tracheal | tracheal | | |
| Size | Cricoid | ring) | ring) | ring) | ring) | T1-T2 | T2-T3 |
| 3.0 | — | 2 (40%) | — | — | — | 2 (40%) | 1 (20%) |
| 3.5 | 1 (10%) | 2 (20%) | 5 (50%) | 1 (10%) | — | 1 (10%) | — |
| 4.0 | 1 (12.5%) | 3 (37.5%) | 4 (50%) | — | — | — | — |
| 4.5 | 3 (20%) | 4 (26.7%) | 3 (20%) | 4 (26.7%) | — | 1 (6.7%) | — |
| 5.0 | 1 (11.1%) | 4 (44.4%) | 2 (22%) | 1 (11.1%) | 1 (11%) | — | — |
| 5.5 | 2 (100%) | — | — | — | — | — | — |
| 6.0 | — | 1 (100%) | — | — | — | — | — |

Analysis of the results showed that when intubated with the device 100, the cuff 108 was positioned below the cricoid in all 50 patients. In two of the patients, the cuff 108 was deeper than anticipated, and the device 100 had to be repositioned. Even after repositioning the device 100 to position distal tip 122 proximal to the patient's carina, the cuff 108 remained below the cricoid outlet in both of these patients. The results further showed that in a majority of patients, the proximal end 132 of the cuff 108 lies between 1^{st} and 2^{nd} tracheal rings when using the marker 124 of the device 100 during intubation. The distal tip 122 was inside the trachea, with no right or left main-stem intubations.

While the disclosure is described herein, using a limited number of embodiments, these specific embodiments are not intended to limit the scope of the disclosure as otherwise described and claimed herein. The precise arrangement of various elements and order of the steps of articles and methods described herein are not to be considered limiting. For instance, although the steps of the methods are described with reference to sequential series of reference signs and progression of the blocks in the figures, the method can be implemented in an order as desired.

1. A device for a trachea, comprising:
a tubular body having a proximal end and a distal end opposite the proximal end, the distal end having A) an opening for ventilation, and B) a distal tip;
a cuff disposed around the tubular body and configured to be inflated to seal the cuff against a wall of a trachea, the cuff being pear-shaped and having a distal end and a tapered proximal end opposite the distal end; and
a plurality of markings disposed on an outer surface of the tubular body, the markings configured to allow the device to be placed in the trachea such that the cuff is positioned below the cricoid of the trachea.

2. The device according to claim 1, wherein the tubular body further includes an inner diameter of about 3.0 mm to about 6.0 mm.

3. The device according to claim 1, wherein the tubular body further includes an outer diameter of about 4.2 mm to about 8.2 mm.

4. The device according to claim 1, wherein the plurality of markings depict vocal cord markings based on customized anatomical measurements of a patient's airways.

5. The device according to claim 1, wherein the plurality of markings are further configured to position the distal tip above the patient's carina.

6. The device according to claim 1, wherein the plurality of markings have a width of about 10.0 mm-15.0 mm.

7. The device according to claim 1, wherein the distance between the plurality of markings and the distal tip range from about 28.0 mm to about 66.0 mm.

8. The device according to claim 1, wherein the tapered proximal end of the cuff is further configured to prevent pressure injury if the cuff is in the cricoid.

9. The device according to claim 1, wherein the cuff has a length of about 8.0 mm to about 18.0 mm.

10. The device according to claim 1, wherein the distance between the plurality of markings and the proximal end of the cuff is about 12.0 mm to about 28.0 mm.

11. The device according to claim 1, wherein the distance between the plurality of markings and the distal end of the cuff may range from about 20.0 mm to about 46.0 mm.

12. The device according to claim 1, wherein the distance between the distal end of the cuff and the distal tip may range from about 8.0 mm to about 20.0 mm.
